(19) United States
(12) Patent Application Publication
(10) Pub. No.: US 2020/0346027 A1
(43) Pub. Date: Nov. 5, 2020

(54) SYSTEMS, METHODS AND APPARATUS FOR STEERING OF ENERGY DEPOSITION IN A DEEP REGIONAL HYPERTHERMIA

(71) Applicant: NEOTHERMA ONCOLOGY, INC., Wichita, KS (US)

(72) Inventors: Charles Anderson, Lee's Summit, MO (US); Anna-Maria Ilisiu, Toronto (CA)

(21) Appl. No.: 16/960,039

(22) PCT Filed: Jan. 8, 2019
(86) PCT No.: PCT/US2019/012712

§ 371 Date: Jul. 3, 2020

Related U.S. Application Data
(60) Provisional application No. 62/614,993, filed on Jan. 8, 2018.

(51) Int. Cl. A61N 1/40; A61N 1/08; A61N 2/02

(52) U.S. Cl. CPC A61N 1/403; A61N 2/02; A61N 1/086

(57) ABSTRACT

The present invention provides, inter alia, apparatus and systems for generating multiple H-fields to additively combine and control deep E-field generation resulting in clinically acceptable centralized heating, and methods for inducing locoregional hyperthermia in a subject using the same. The methods, systems and apparatus disclosed herein provide improved hyperthermia treatment to subjects in need thereof, such as, for example, subjects suffering from cancer.

### CROSS REFERENCE TO RELATED APPLICATIONS

The present application claims benefit of U.S. Provisional Patent Application Ser. No. 62/614,993, filed on Jan. 8, 2018 which application is incorporated by reference herein in its entirety.

### FIELD OF THE INVENTION

The present invention relates to inducing locoregional hyperthermia with deep centralized heating by using inductively coupled coil pairs such that multiple magnetic fields are created to additively combine E-fields between them. This invention allows an inductively coupled system to target the E-fields irrespective of the permittivity of the tissue and avoid generation of primarily superficial heating of muscle tissue. This invention allows the use of a simple, cost effective design that does not require broadband RF generators or phase-controlled antenna.

### BACKGROUND OF THE INVENTION

Targeted deep hyperthermia is known for therapeutic applications such as cancer treatment, tumor ablation and treatment of other diseases (Anderson et al., U.S. Patent Application Publication No. 20180015294 A1). A system for RF hyperthermia based on inductively coupled (H-field) antennae allow for advantages over other systems that primarily heat with radiated E-fields or coupled E-fields. The magnetic permeability of all relevant tissues is close to 1, so predicting the field pattern of the H-field is known to be highly accurate. Due to the orientation and method of generating E-fields for heating, preferential heating in fatty tissue can be avoided with a properly designed inductively coupled system.

Current methods of heating with H-field have lacked the ability to heat deeply, i.e., to desired clinical depths in large patients of more than 10 cm. The present invention relates to systems and methods to address these issues identified above.

### SUMMARY OF THE INVENTION

The present invention demonstrated that, creating targeted heating in an inductively coupled system is not trivial. Conventional systems in this field, including adaptations of inductively coupled systems have failed to heat at clinically relevant depths of greater than 10 cm because methods used to control the resulting E-field locations have not been available and/or used. In simulation of other inductive systems around a virtual human model of an abdomen, systems utilizing surface pancake coils, circumferential coil windings, and Helmholtz-based circumferential coil pairs could not generate acceptable heating at depth because E-field generation patterns would always be limited by large energy deposition in the skeletal and abdominal muscles. The present invention uses multiple H-fields to create additive E-fields (primarily through generation of eddy currents) which can achieve clinically relevant heating at low frequencies without increasing the complexity or cost of a system by methods such as phase control. In particular, the design allows for relevant deep heating at open Industrial, Scientific, and Medical frequencies of 13.56 MHz, 27.1 MHz, and 40.68 MHz which do not respond well to phase-controlled antennas at the relevant geometry of humans or large animals. In addition, the present system is MRI-safe and transparent, allowing for simultaneous operation in order to take advantage of techniques such as magnetic resonance thermographic imaging.

In one embodiment of the present invention, a system for generating multiple H-fields to additively combine and control deep E-field generation resulting in clinically acceptable centralized heating is provided. This system comprises:
a) a base of sufficient dimensions to accommodate a subject;
b) one or more sectors, wherein:
   i. each sector comprises at least one pair of coils, wherein each coil of the pair is substantially parallel to and faces the other coil and each coil of the pair is separated by a distance;
   ii. each coil of the pair is arranged in a predetermined design; and
   iii. the width of each coil is equal to or greater than the separation distance between each coil pairs;
   and
c) one or more radio-frequency (RF) generators to control the amplitude of

RF applied on each coil of the pair to move the Specific Absorption Rate (SAR) pattern in the desired direction.

In another embodiment of the present invention, a method for inducing locoregional hyperthermia in a subject in need thereof is provided. This method comprises:
a) providing an apparatus for generating multiple H-fields to additively combine and control deep E-field generation, the apparatus comprising:
   (I) a base to accommodate the subject;
   (II) one or more sectors, wherein:
      i. each sector comprises at least one pair of coils, wherein each coil of the pair is substantially parallel to and faces the other coil and each coil of the pair is separated by a distance;
      ii. each coil of the pair is arranged in a predetermined design; and
      iii. the width of each coil is equal to or greater than the separation distance between each coil pairs;
      and
   (III) one or more radio-frequency (RF) generators to control the amplitude of RF applied on each coil of the pair to move the Specific Absorption Rate (SAR) pattern in the desired direction;
b) adjusting the size of coils in each sector and the size of the base based on the size of the subject and the desired depth of heating;
c) placing the subject on the base; and
d) generating multiple H-fields to create targeted heating at the desired depth in the subject.

In an additional embodiment of the present invention, a system for generating multiple H-fields to additively combine and control deep E-field generation resulting in clinically acceptable centralized heating is provided. This system comprises:
a) an interchangeable base;
b) one or more sectors, wherein:
   i. each sector comprises at least one pair of coils, wherein each coil of the pair is substantially parallel to and faces the other coil and each coil of the pair is separated by a distance;
   ii. each coil of the pair is arranged in a predetermined design;
   iii. the width of each coil is equal to or greater than the separation distance between each coil pairs; and
   iv. the length of each coil is equal to or greater than 1.5 times the separation distance between each coil pairs;
c) one or more radio-frequency (RF) generators to control the amplitude of RF applied on each coil of the pair to move the Specific Absorption Rate (SAR) pattern in the desired direction
d) a solid-state switch;
e) an air-cooling setup; and optionally
f) an MRTI setup.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a single coil arranged so that two H-fields are generated in opposite phase (in the hidden axis). The coil can be fed at any point but practically so on one of the longest edges of the left or right side. This electromagnetic design is also accomplished with two coils, one in each current rotation direction and overlaid closely.

FIG. 2 is a model depiction that shows a sector (1) containing a pair of coils (10a and 10b) from FIG. 1, separated by a distance of (20) (e.g., 25 cm), with e.g., 20 cm of muscle tissue placed between. As shown, the width (30a, 30b) of each coil is equal to the separation distance (20) between each coil pair (10a, 10b).

FIG. 3 is the resulting H-field plot (symmetrical slice) as solved with an EM-FDTD software, showing two opposite phase H-fields generated by a coil pair.

FIG. 4 is the resulting E-field plot (symmetrical slice) as solved with an EM-FDTD software, showing the eddy currents generated, and especially the additive region in the center by a coil pair.

FIG. 5A is a schematic diagram showing that the system utilizes digital control signals (blue) to command a solid state switch and one or more RF generators to power (black) and switch in discrete time slices and amplitude the selection of three sectors of the dual H-field inductively coupled coil pairs.

FIG. 5B is a cartoon of an exemplary system of the present invention. The system utilizes a “base” (10) containing a MRI-safe solid-state switch (not shown) and RF traps (not shown), swappable to an “applicator” (30) of different sizes of RF (not shown) and MRI coils (not shown) to conform to different subject anatomy. Also shown, grooves (20) for subject cooling and comfort with pressurized air, to be diffused through a replaceable foam pad (see FIG. 6D, in light blue).

FIGS. 6A to 6D show the construction of an air-cooling setup.

FIG. 7A is a schematic diagram showing the system setup.

FIG. 7B shows the radio-frequency (RF) generators.

FIG. 7C shows the arrangement of butterfly coils and a box containing ground pork to simulate human tissue.

FIG. 8 shows the axis for the temperature probe placement.

FIG. 9 shows the result of 1-inch depth test.

FIG. 10 shows the result of 4-inch depth test.

FIG. 11 shows the result of center line test.

### DETAILED DESCRIPTION OF THE INVENTION

The present invention discloses a system for generating multiple H-fields to additively combine and control deep E-field generation resulting in clinically acceptable centralized heating, and methods for inducing locoregional hyperthermia in a subject using the same.

One embodiment of the present invention is a system for generating multiple H-fields to additively combine and control deep E-field generation resulting in clinically acceptable centralized heating. This system comprises:
a) a base of sufficient dimensions to accommodate a subject;
b) one or more sectors, wherein:
   i. each sector comprises at least one pair of coils, wherein each coil of the pair is substantially parallel to and faces the other coil and each coil of the pair is separated by a distance;
   ii. each coil of the pair is arranged in a predetermined design; and
   iii. the width of each coil is equal to or greater than the separation distance between each coil pairs;
   and
c) one or more radio-frequency (RF) generators to control the amplitude of RF applied on each coil of the pair to move the Specific Absorption Rate (SAR) pattern in the desired direction.

As used herein, “Specific Absorption Rate” or “SAR” refers to is a measure of the rate at which energy is absorbed by the human body when exposed to a radio frequency (RF) electromagnetic field. It can also refer to absorption of other forms of energy by tissue, including ultrasound. It is defined as the power absorbed per mass of tissue and has units of watts per kilogram (W/kg). An SAR greater than 20 W/kg is considered clinically relevant.

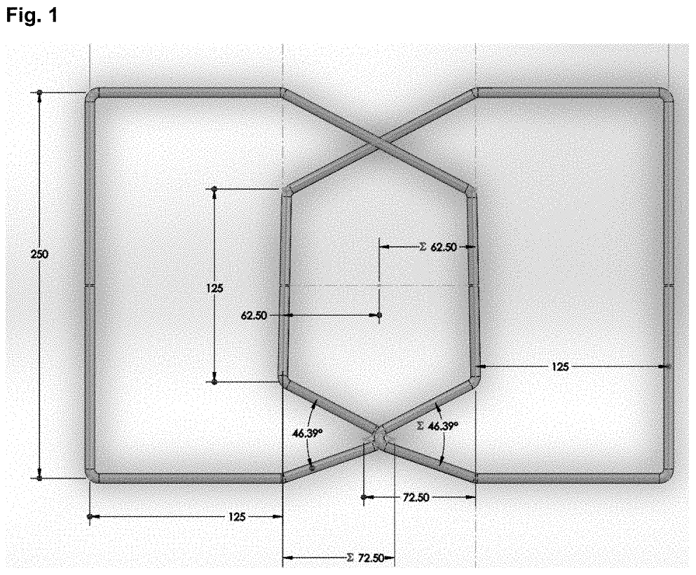

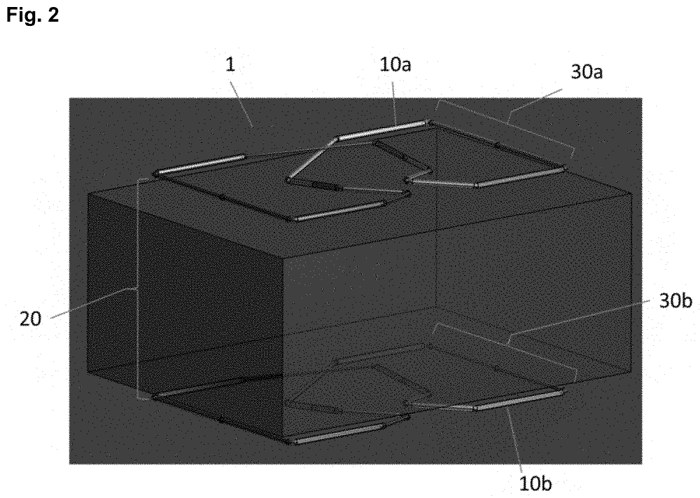

In some embodiments, the predetermined design is configured in the form of a “butterfly” substantially as shown in FIG. 1 and FIG. 2. While, a “butterfly” configuration is depicted and described herein, the coil pairs may be configured in other shapes, so long as the desired deep heating effects are obtained. With respect to the “butterfly” configuration, in certain embodiments, one half of the butterfly is overlapped with the other half. In certain embodiments, one half of the butterfly is overlapped 50% with the other half, thereby the length of the coil is equal to 1.5 times the separation distance between coil pairs. In certain embodiments, each single coil comprises two individual coils, for example, being manufactured as two coils, and powered with 0° and 180°, respectively, to achieve the same function.

In some embodiments, the system further comprises a switching setup previously described in Anderson et al., U.S. Ser. No. 15/653,462 filed Jul. 18, 2017, which is hereby incorporated by reference herein. By incorporating such a switching setup, the present invention allows for a highly cost effective and competitive system that contains one or more RF generators, such as 1-10 or preferably 1, 2, 3, or 4 RF generators and switching networks to control the pairs of coils and create selective heating at depth. In certain embodiments, the switching setup comprises a solid-state switch that selects the sectors and amplitude in discrete time slices to heat desired regions while avoiding inadvertent hotspot generation.

In some embodiments, the system further comprises a magnetic resonance thermographic imaging (MRTI) system for treatment monitoring, adjustment, and reporting.

In some embodiments, each sector further comprises one or more series-tuning capacitors placed along the length of one or more coils in order to increase the homogeneity of the field and reduce the magnitude of the radiated E-field, and improve tuning.

In some embodiments, the size of the base is adjustable/interchangeable, and the size of the coils is adjustable/interchangeable, in order to treat subjects of different size, or to provide additional therapeutic treatment options, while minimizing component cost.

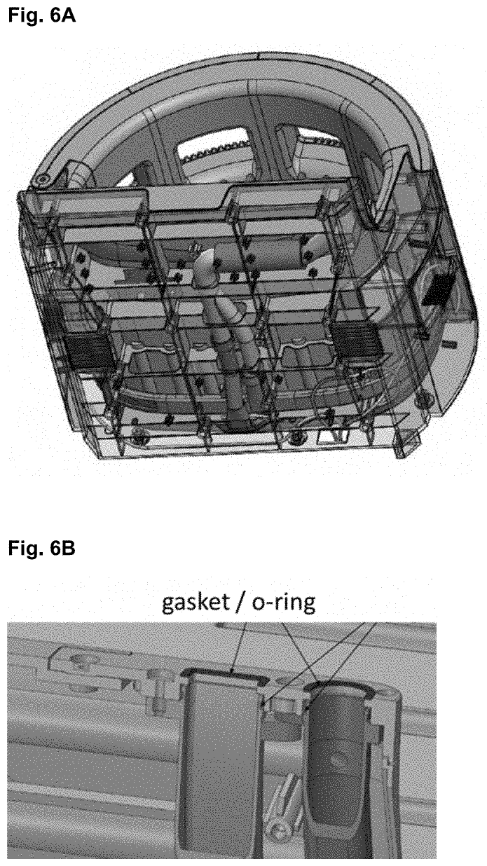

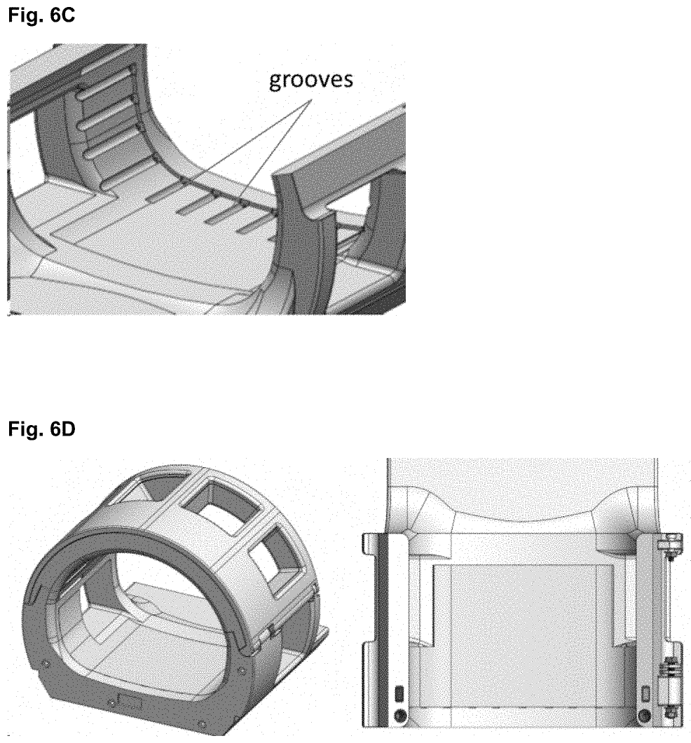

In some embodiments, the system further comprises an air-cooling setup through a membrane in, e.g., the base, in order to maximize subject comfort throughout the procedure. An exemplary air-cooling setup can be, e.g., a diffuse air-cooling foam, which is open-celled with a packing density no greater than 0.5 and with sufficient modulus to support the subject. The air-cooling setup is significant in RF hyperthermia because it takes up minimal space in the MRI room and thus minimally impacts the normal MRI workflow. Air is brought in from outside and passes through a 2″ waveguide. The main routing for the air is as follow: the air is split into subject cooling (dark blue) and electrical component cooling (light blue) (FIG. 6A); it is sealed at the hinge of the device via gaskets and o-rings (FIG. 6B, in black); the air is designed to be appropriate air-speed to provide a “cool” sensation using the Berkeley comfort model (see https://www.cbe.berkeley.edu/research/briefs-thermmodel.htm); the air is then sent into grooves (FIG. 6C), which are diffused through a disposable insert foam pad (FIG. 6D, in light blue).

Another embodiment of the present invention is a method for inducing locoregional hyperthermia in a subject in need thereof. This method comprises:
a) providing an apparatus for generating multiple H-fields to additively combine and control deep E-field generation, the apparatus comprising:
   (I) a base to accommodate the subject;
   (II) one or more sectors, wherein:
      i. each sector comprises at least one pair of coils, wherein each coil of the pair is substantially parallel to and faces the other coil and each coil of the pair is separated by a distance;
      ii. each coil of the pair is arranged in a predetermined design; and
      iii. the width of each coil is equal to or greater than the separation distance between each coil pairs;
      and
   (III) one or more radio-frequency (RF) generators to control the amplitude of RF applied on each coil of the pair to move the Specific Absorption Rate (SAR) pattern in the desired direction;
b) adjusting the size of coils in each sector and the size of the base based on the size of the subject and the desired depth of heating;
c) placing the subject on the base; and
d) generating multiple H-fields to create targeted heating at the desired depth in the subject.

As used herein, a “subject” is a mammal, preferably, a human. In addition to humans, categories of mammals within the scope of the present invention include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

In some embodiments, the desired depth of heating is greater than 2 cm, greater than 5 cm, greater than 10 cm, greater than 15 cm, greater than 20 cm, greater than 25 cm, greater than 30 cm, greater than 35 cm, greater than 40 cm, greater than 45 cm or greater than 50 cm. In certain embodiments, the desired depth of heating is around 55 cm.

Yet another embodiment of the present invention is a system for generating multiple H-fields to additively combine and control deep E-field generation resulting in clinically acceptable centralized heating. This system comprises:
a) an interchangeable base;
b) one or more sectors, wherein:
   i. each sector comprises at least one pair of coils, wherein each coil of the pair is substantially parallel to and faces the other coil and each coil of the pair is separated by a distance;
   ii. each coil of the pair is arranged in a predetermined design;
   iii. the width of each coil is equal to or greater than the separation distance between each coil pairs; and
   iv. the length of each coil is equal to or greater than 1.5 times the separation distance between each coil pairs;
c) one or more radio-frequency (RF) generators to control the amplitude of RF applied on each coil of the pair to move the Specific Absorption Rate (SAR) pattern in the desired direction
d) a solid-state switch;
e) an air-cooling setup; and optionally
f) an MRTI setup.

In some embodiments, the radio-frequency (RF) applied in the systems and methods disclosed herein can be selected from 13.56 MHz, 27.1 MHz, or 40.68 MHz.

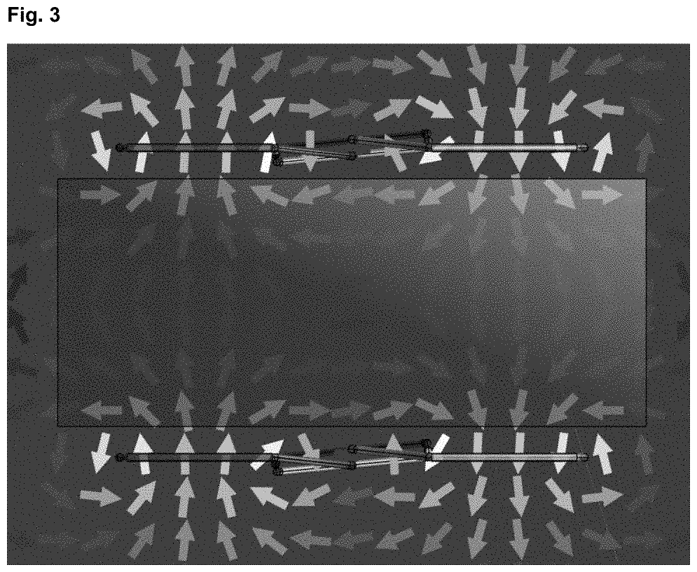

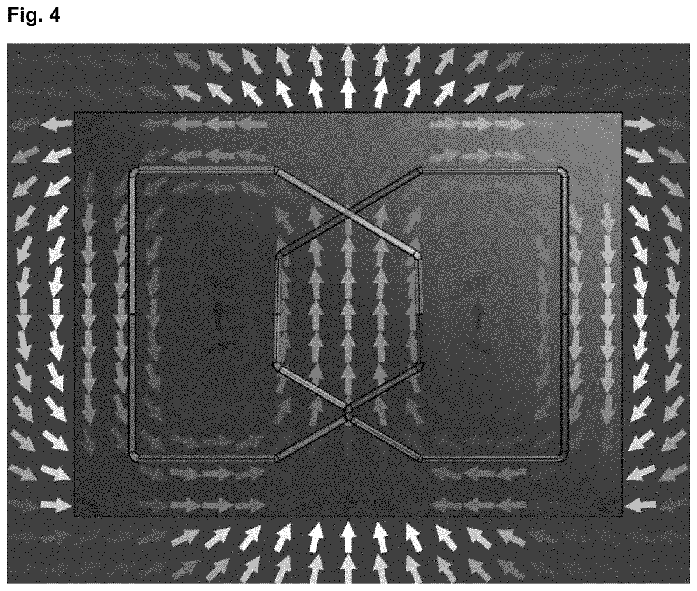

Another embodiment of the present invention is an apparatus substantially as disclosed in FIGS. 1 to 5B.

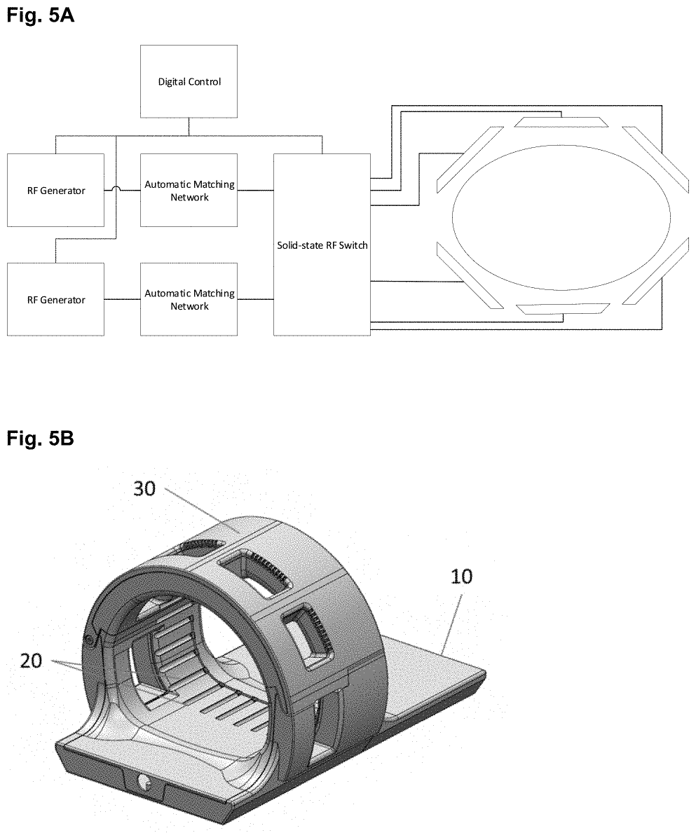

Another embodiment of the present invention is a system substantially as disclosed in FIG. 5A.

### EXAMPLES

The invention is further illustrated by the following examples, which are offered for illustrative purposes, and are not intended to limit the invention in any manner. Those of skill in the art will readily recognize a variety of noncritical parameters, which can be changed or modified to yield essentially the same results.

### Example 1

### System Setup and Results

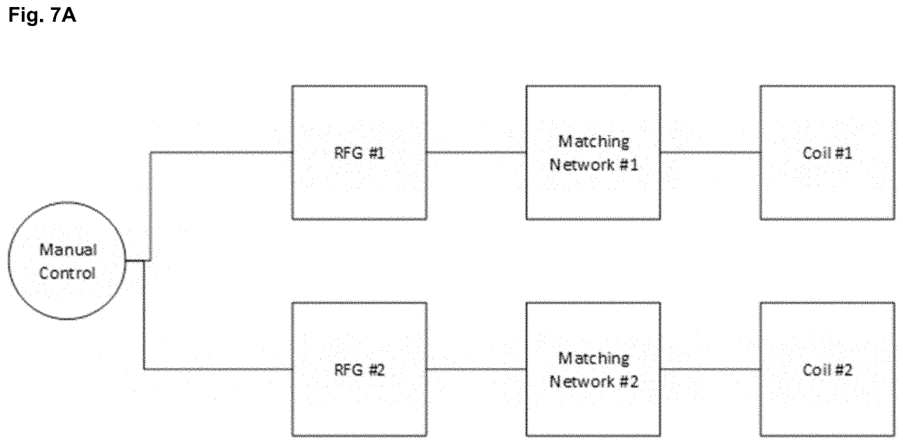

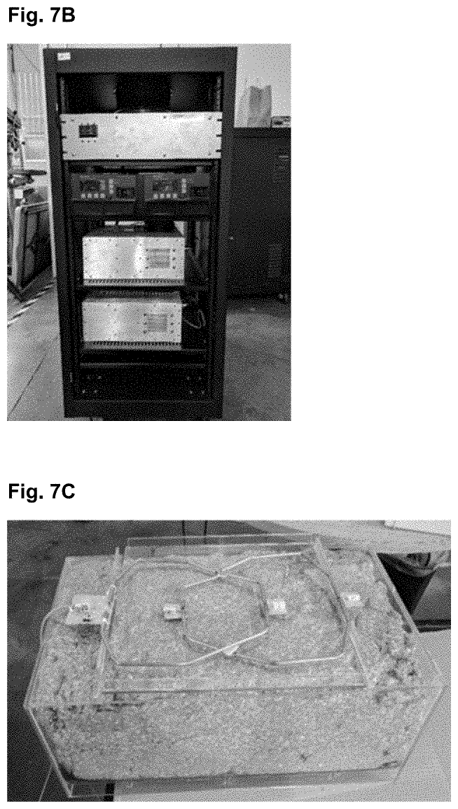

The system was connected as shown in FIG. 7A. The coils were powered by 2 radiofrequency generators that are phase locked 180 degrees (FIG. 7B). Each generator had a matching network connected in line to the coil. Two butterfly coils were attached at the top and bottom of the meat box (FIG. 7C). The box was filled with 100 pounds of ground pork (˜10-15% fat content) to simulate human tissue. Power meters for each generator were connected after matching networks for SWR and Power measurement.

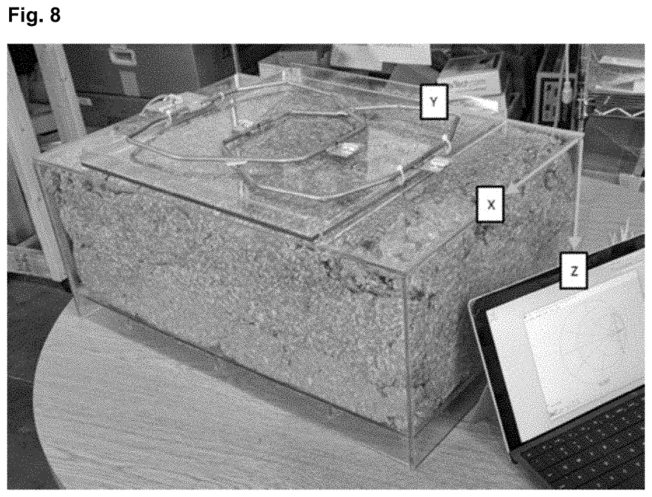

Probes were placed using a depth introducer to control placement of the temperature point. Temperature was measured with a fiber optic temperature system that is immune to electromagnetic fields. The axis for the probe placement points is show in FIG. 8. Each positive axis is shown. For each test, probe position was saved using the axis shown.

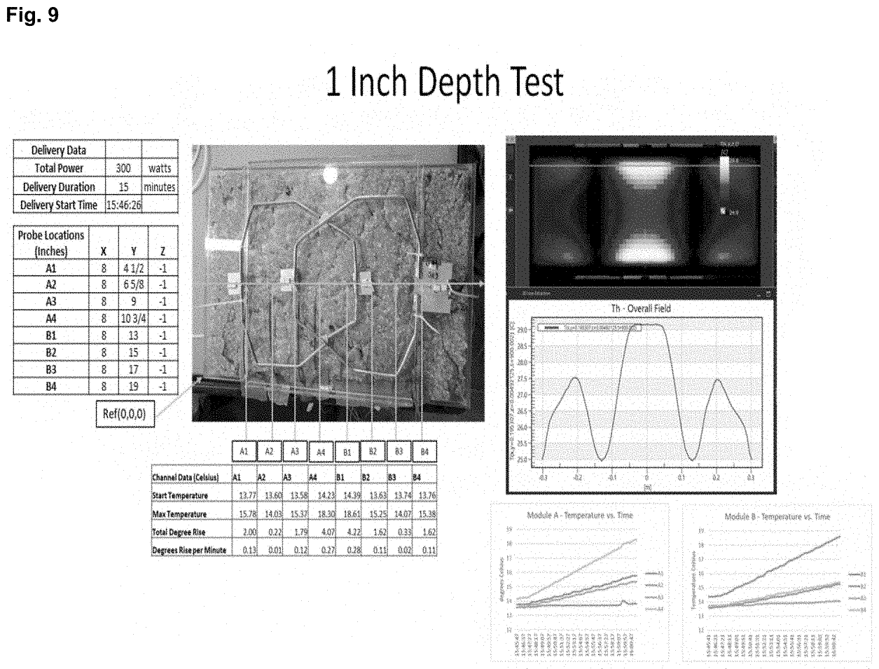

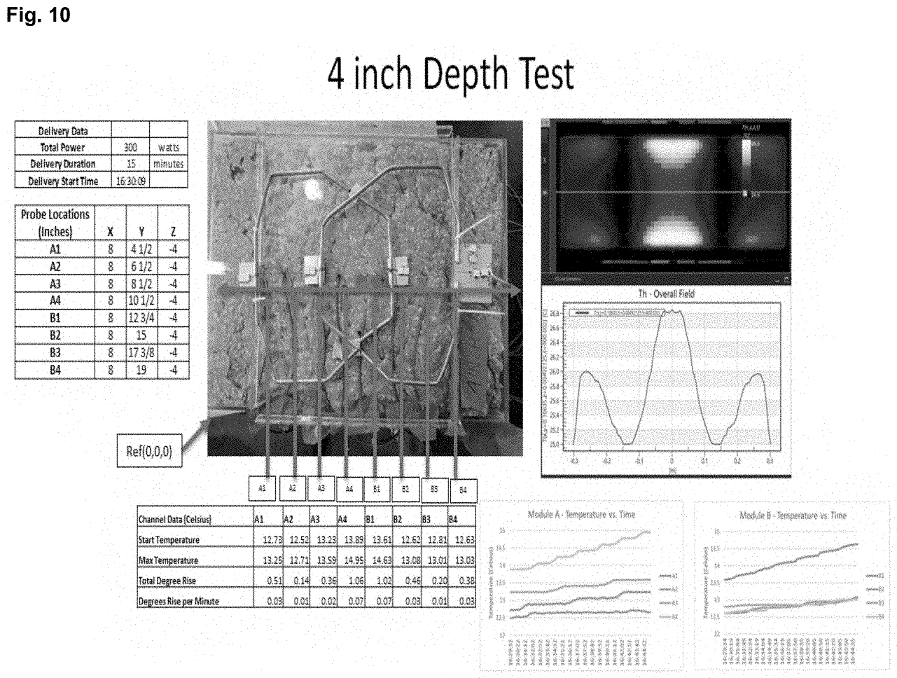

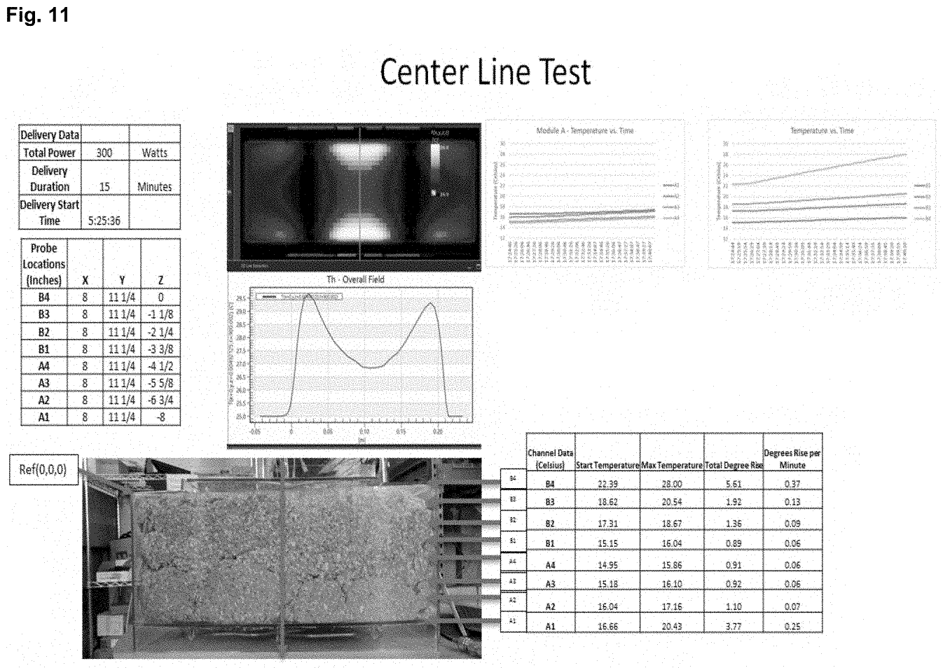

The test results were recorded and shown in FIG. 9 (one-inch depth test), FIG. 10 (four-inch depth test) and FIG. 11 (center line test). The actual temperature results were in good agreement with the thermal simulation.

All patents, patent applications, and publications cited above are incorporated herein by reference in their entirety as if recited in full herein.

The invention being thus described; it will be obvious that the same may be varied in many ways. Such variations are not to be regarded as a departure from the spirit and scope of the invention and all such modifications are intended to be included within the scope of the following claims.

What is claimed is:

1. A system for generating multiple H-fields to additively combine and control deep E-field generation resulting in clinically acceptable centralized heating, comprising:
a) a base of sufficient dimensions to accommodate a subject;
b) one or more sectors, wherein:
i. each sector comprises at least one pair of coils, wherein each coil of the pair is substantially parallel to and faces the other coil and each coil of the pair is separated by a distance;
ii. each coil of the pair is arranged in a predetermined design; and
iii. the width of each coil is equal to or greater than the separation distance between each coil pairs; and
c) one or more radio-frequency (RF) generators to control the amplitude of RF applied on each coil of the pair to move the Specific Absorption Rate (SAR) pattern in the desired direction.

2. The system of claim 1, wherein the predetermined design is configured in the form of a butterfly.

3. The system of claim 2, wherein one half of the butterfly is overlapped with the other half.

4. The system of claim 2, wherein one half of the butterfly is overlapped 50% with the other half, thereby the length of the coil is equal to 1.5 times the separation distance between each coil pair.

5. The system of claim 1, wherein each coil of a pair comprises two individual coils.

6. The system of claim 1, further comprising a switch.

7. The system of claim 6, wherein the switch is a solid-state switch that selects the sectors and amplitude in discrete time slices.

8. The system of claim 1, further comprising a magnetic resonance thermographic imaging (MRTI) setup for treatment monitoring, adjustment, and reporting.

9. The system of claim 1, wherein each sector further comprises one or more series-tuning capacitors placed along the length of each coil.

10. The system of claim 1, wherein the base and coil pairs are variable to accommodate different subject sizes and therapeutic applications.

11. The system of claim 1, further comprising an air-cooling setup.

12. The system of claim 1, wherein the RF applied is selected from 13.56 MHz, 27.1 MHz, or 40.68 MHz.

13. A method for inducing locoregional hyperthermia in a subject in need thereof, comprising:
a) providing an apparatus for generating multiple H-fields to additively combine and control deep E-field generation, the apparatus comprising:
(I) a base to accommodate the subject;
(II) one or more sectors, wherein:
i. each sector comprises at least one pair of coils, wherein each coil of the pair is substantially parallel to and faces the other coil and each coil of the pair is separated by a distance;
ii. each coil of the pair is arranged in a predetermined design; and
iii. the width of each coil is equal to or greater than the separation distance between each coil pairs; and
(III) one or more radio-frequency (RF) generators to control the amplitude of RF applied on each coil of the pair to move the Specific Absorption Rate (SAR) pattern in the desired direction;
b) adjusting the size of coils in each sector and the size of the base based on the size of the subject and the desired depth of heating;
c) placing the subject on the base; and
d) generating multiple H-fields to create targeted heating at the desired depth in the subject.

14. The method of claim 13, wherein the subject is a mammal.

15. The method of claim 14, wherein the mammal is selected from the group consisting of humans, veterinary animals, and agricultural animals.

16. The method of claim 13, wherein the subject is a human.

17. The method of claim 13, wherein the desired depth of heating is greater than 10 cm.

18. The method of claim 13, wherein the RF applied is selected from 13.56 MHz, 27.1 MHz, or 40.68 MHz.

19. A system for generating multiple H-fields to additively combine and control deep E-field generation resulting in clinically acceptable centralized heating, comprising:
a) an interchangeable base;
b) one or more sectors, wherein:
i. each sector comprises at least one pair of coils, wherein each coil of the pair is substantially parallel to and faces the other coil and each coil of the pair is separated by a distance;
ii. each coil of the pair is arranged in a predetermined design;
iii. the width of each coil is equal to or greater than the separation distance between each coil pairs; and
iv. the length of each coil is equal to or greater than 1.5 times the separation distance between each coil pairs;
c) one or more radio-frequency (RF) generators to control the amplitude of RF applied on each coil of the pair to move the Specific Absorption Rate (SAR) pattern in the desired direction
d) a solid-state switch;
e) an air-cooling setup; and optionally
f) an MRTI setup.

20. The system of claim 19, wherein the RF applied is selected from 13.56 MHz, 27.1 MHz, or 40.68 MHz.

21. An apparatus substantially as disclosed in FIGS. 1 to 5B.

22. A system substantially as disclosed in FIG. 5A.
